(19) United States
(12) Patent Application Publication
(10) Pub. No.: US 2024/0059739 A1
(43) Pub. Date: Feb. 22, 2024

(54) PEPTIDES FOR INCREASING DRUG DELIVERY TO CANCER

(71) Applicant: Chapman University, Orange, CA (US)

(72) Inventors: Sun Yang, Orange, CA (US); Kamaljit Kaur, Orange, CA (US)

(21) Appl. No.: 18/330,748

(22) Filed: Jun. 7, 2023

Related U.S. Application Data
(60) Provisional application No. 63/349,884, filed on Jun. 7, 2022.

(51) Int. Cl. C07K 7/08; A61K 31/47; A61K 31/704; A61P 35/00

(52) U.S. Cl. CPC C07K 7/08; A61K 31/47; A61K 31/704; A61P 35/00

(57) ABSTRACT

Disclosed herein are tumor-targeting peptides and methods of treating cancer with the combination of a tumor-targeting peptide and a chemotherapeutic agent.

### CROSS-REFERENCE TO RELATED APPLICATION

This application claims the benefit of, and priority to, U.S. Provisional Patent Application 63/349,884, filed Jun. 7, 2022, the entire contents of which is incorporated by reference herein.

### SEQUENCE LISTING

A Sequence Listing is submitted herewith and incorporated by reference herein as an XML file created on Jun. 6, 2023, entitled “1959206-00035_Sequence_Listing.xml” and having a size of 52 KB.

### BACKGROUND

Human cutaneous melanoma (CM) is one of the few cancers in which the incidence rate continues to increase; thereby, making this disease a rising public health concern in the United States. The advances in the field of molecularly targeted therapeutics and immunotherapy have changed the landscape of melanoma management and significantly improved patient survival. However, the rapid development of drug resistance to targeted therapy and the fact that only a subset of melanoma patients respond to immunotherapy, has led researchers to investigate new and improved strategies for melanoma treatment.

Compared to many human malignancies, CM is highly resistant to traditional cytotoxic chemotherapy. The only FDA-approved cytotoxic drugs for melanoma treatment are dacarbazine and temozolomide with very limited efficacy. Although doxorubicin (DOX) is highly effective in the treatment of many types of cancer, melanoma is resistant to its cytotoxic effects as a result of the intrinsic resistance of this cancer type to DOX. Similar resistance was also reported with cisplatin treatment in melanoma. An enzyme, neuronal nitric oxide synthase (nNOS) was found to be overexpressed in CM and has been identified as a key player in melanoma-genesis by enhancing tumor growth and interferon-gamma-stimulated melanoma progression.

Previous studies demonstrated that novel nNOS inhibitors, such as MAC-3-190 and HH044, exhibit promising anti-melanoma activities by inhibiting nNOS-mediated nitric oxide signaling (Tong et al. Sci Rep 12:1701, 2022; Yang et al. Antioxid Redox Signal 19:433-447, 2013; Huang et al. J Med Chem 57:686-700, 2014; Cinelli et al. J Med Chem 60:3958-3978, 2017).

The mechanism of drug resistance in melanoma is complex. Increased drug efflux is one of the most observed mechanisms, resulting in reduced intracellular drug levels suboptimal for cytotoxicity. A practical approach to overcoming drug resistance is to improve drug delivery and, as a result, increase intracellular drug accumulation and efficacy. Different strategies are used to increase the uptake of drugs and prevent drug efflux, such as the use of drug carriers like liposomes or nanoparticles, drug conjugates where the drug is covalently conjugated to a targeting ligand (e.g. peptides), and co-administration of drugs with targeting ligands.

While drug carriers and drug conjugates have been extensively studied, the use of co-administration of drugs with a cancer cell-targeting ligand is less explored. DOXIL®, a liposomal formulation of chemotherapeutic DOX, is used clinically. In addition, a peptide-drug conjugate and several antibody-drug conjugates are now approved for cancer treatment. These drug conjugates target different cell-surface receptors overexpressed in cancer cells for specific receptor mediated uptake of the conjugate. Co-administration of a targeting ligand with a drug also leads to increased uptake and efficacy. For instance, co-administration of the peptide iRGD with various cytotoxic agents, like doxorubicin, nab-paclitaxel (nanoparticles), or trastuzumab (antibody), enhanced the therapeutic efficacy of each of them. This later strategy has advantages such as no requirement for drug modification which can reduce drug activity, and large amounts of drug can be delivered into the tumor tissue due to bystander effects. Novel approaches to improve available melanoma therapeutics, including organic and inorganic nanomaterials, have been developed for drug delivery, such as liposomes, polymers, dendrimers, and micelles. Different nanomaterials offer various advantages, including controlled release, reduced systemic toxicity, and protection from metabolic inactivation. The use of nanoparticles, however, has been limited due to concerns regarding in vivo distribution, immunogenicity, limited tissue penetration and stability, rapid removal, and degradation.

In recent years, more efforts have focused on conjugation to cancer cell targeting ligands such as monoclonal antibodies (mAbs) and peptides to improve targeted delivery of anti-cancer drugs or nanoparticles. mAbs are attractive delivery vehicles due to their high target specificity and affinity. However, mAbs possess significant limitations. Due to their complexity and size, mAbs have limited tissue penetration, high production costs, and a high risk of immunogenicity. In addition to target expression and internalization, drug loading and conjugation are essential factors to consider in antibody-drug conjugates (ADCs). Different ratios have been found to affect the pharmacokinetic properties, therapeutic index, and antigen-binding.

### SUMMARY

Disclosed herein are methods for improving drug delivery, for example drug delivery to cancer cells.

Disclosed herein are compositions comprising tumor-targeting peptides and anticancer drugs.

Disclosed methods comprise co-administration of a melanoma cell-specific peptide with a chemotherapeutic agent such as DOX or MAC-3-190 to improve their uptake by melanoma cells.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows the structures of doxorubicin and MAC-3-190.

FIG. 2A is a schematic showing peptide array screening for melanoma specific peptides. The average CyQUANT fluorescence intensity of the peptide bound cells was used to estimate the relative cells bound to the peptides.

FIG. 2B shows relative fluorescence compared to normal human embryonic kidney cells (HEK-293). The top peptides (such as peptide 11, black) showing a better binding profile for melanoma cells were further labeled with FITC for cellular uptake analysis.

FIG. 2C shows relative fluorescence compared to normal human embryonic kidney cells (A375 cells). The top peptides (such as 11, black) showing a better binding profile for melanoma cells were further labeled with FITC for cellular uptake analysis.

FIG. 3A shows a representative fluorescence microscopy image of A375 melanoma cells after incubation with FITC-KK-11 (0.5 μM) for 30 min at 37° C. Cell nuclei were stained blue with DAPI.

FIG. 3B shows uptake of FITC-peptides KK-1, KK-11, KK-12, and KK-13 by human melanoma cells. The bar represents the average fluorescence density of three cell lines (A375, Sk-mel-28, and wm3211 cells) detected by fluorescence-activated cell sorting (FACS) analysis. The cells were incubated with FITC-labeled peptide for 30 min at 37° C. (0.5 μM, grey bar; 1 μM, dark bar). The control is cells incubated with FITC alone. The bar graph shows mean fluorescence intensity (MFI) of three experiments ±SD; *, p<0.05 compared to 0.5 μM FITC alone; #, p<0.001 compared to 1 μM FITC alone. FIG. 3C shows co-treatment of KK-11 peptide enhanced the cytotoxicity of DOX in melanoma A375 cells. Cells were either treated with DOX or co-treated with DOX and KK-11 (1 μM) for 72 h (KK-11+DOX) or pretreated with KK-11 for 4 h, then washed away following a DOX 72 h treatment (KK-11 to DOX). Control is DMSO or KK-11 alone. Cell viability was detected by a MTT colorimetric assay. Statistical analysis was performed with a student's t-test, and error bars denote mean±SD; **, p<0.01; ***, p<0.001 compared to DOX alone. D) Co-treatment of KK-11 did not enhance the cytotoxicity of nNOS inhibitor MAC-3-190 in A375 melanoma cells. Cells were treated with MAC-3-190 at various concentrations with or without 1 μM KK-11 for 72 h. Cell viability was detected by MTT assay. The assays were repeated independently at least three times. FIG. 3D shows co-incubation of KK-11 with MAC-3-190 did not enhance cytotoxicity in vitro.

FIG. 4A and FIG. 4B-1 and FIG. 4B-2 show in vivo and ex vivo organ distribution images of VivoTag 680 XL-labeled D-aa KK-11 in athymic nude mice bearing human melanoma A375 tumors. Saline or VivoTag-KK-11 in a dose of 8 μg per mouse was injected into the mice via the tail vein (n=2 for each time point).

FIG. 4A shows representative images of CT-fluorescence scan at tumor area section were captured in live mice at different times after injection. The top panels represent the scanned images of the tumor section area in coronal views. The bottom-row pictures display the fluorescence image in trans-axial views: arrows point to the spine, and circles mark the xenograft tumors.

FIG. 4B shows at defined time intervals (5 min, 15 min, 30 min, 1 h, 1.5 h, 2 h, 3 h, 4 h, 6 h, 24 h, and 48 h) following peptide injection, animals were euthanized. Organs including tumor xenografts (T), liver (L), spleen (S), heart and lungs (H+L), kidneys (K), and brain (B) were excised and imaged using the IVIS Spectrum imaging system. Representative fluorescence images of different organs show peptide distribution at different time points as indicated.

FIG. 5A and FIG. 5B and FIG. 5C show enhanced antitumor activity of nNOS inhibitor MAC-3-190 co-injected with D-aa KK-11 peptide in a xenograft mouse model. Mice bearing human melanoma A375 tumors were intravenously injected with either D-aa KK-11, MAC-3-90, or a mixture of MAC-3-190 (5 mg/kg) and D-aa KK-11 (1.75 mg/kg) or 1×PBS once daily. FIG. 5A; the tumors were measured three times a week using digital Vernier calipers. Tumor volume (mm³) was calculated as [Length×(Width²)/2. FIG. 5B; the tumors were harvested and weighed after 21 days of treatment (Control: n=10; D-aa KK-11 alone: n=5; MAC-3-190: n=5; D-aa KK-11+MAC-3-190: n=10). FIG. 5C; the body weights of the mice were measured after 21 days of treatment. Statistical analysis was performed with student's t-test and error bars denote mean±SD: *, p<0.05 compared to control group.

### DETAILED DESCRIPTION

Melanoma is the most fatal type of skin cancer and is notoriously resistant to chemotherapy. The response of melanoma to current treatments is difficult to predict.

Peptide-drug conjugates (PDCs) are an appealing alternative to ADCs and may offer solutions to the limitations posed by mAbs. Short peptides have low oral bioavailability but are easily synthesized to homogeneity and allow for considerable flexibility due to the diversity of amino acid combinations, which enable the alteration of physiochemical properties, specificity, and stability, generally without immunogenic responses.

PDCs have shown promising anticancer activities in many difficult-to-treat malignancies, such as pancreatic cancer and melanoma. Peptide-doxorubicin conjugates are being developed to treat triple-negative breast cancer (TNBC) by targeting cell-surface keratin-1 (K1) or epidermal growth factor receptor (EGFR) in cancer cells. The success of PDCs is also evident from the FDA approval (2018) of a peptide conjugate, lutetium 177 DOTA-TATE, where radionuclide ¹⁷⁷Lu is covalently linked to a somatostatin receptor targeting peptide. This PDC is approved for treatment of somatostatin receptor-positive gastroenteropancreatic neuroendocrine tumors.

Disclosed herein are tumor-targeting peptides, compositions comprising the peptides, and methods of treatment comprising administration of tumor-targeting peptides to increase drug delivery to melanoma cells.

### Definitions

The term “about,” as used herein, generally refers to a range of values +/−10% of a specified value. The term “about,” also refers within the pharmaceutically acceptable limits found in the United States Pharmacopeia (USP-NF 21), 2003 Annual Edition, or available at www.usp.org, for amount of active pharmaceutical ingredients. With respect to blood levels, “about” means within FDA acceptable guidelines.

“Administration,” or “to administer” means the step of giving (i.e. administering) a pharmaceutical composition or active ingredient to a subject. The pharmaceutical compositions disclosed herein can be administered via a number of appropriate routs, including oral and intramuscular or subcutaneous routes of administration, such as by injection, topically, or use of an implant.

“Patient” means a human or non-human subject receiving medical or veterinary care.

The phrase “pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic.

Some examples of materials which may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Pharmaceutical composition” means a formulation with an active ingredient. The word “formulation” means that there is at least one additional ingredient (such as, for example and not limited to, an albumin [such as a human serum albumin (HSA) or a recombinant human albumin] and/or sodium chloride) in the pharmaceutical composition in addition to an active ingredient. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Therapeutic formulation” means a formulation that can be used to treat and thereby alleviate a disorder, a disease, and/or a symptom associated thereof.

“Therapeutically effective amount” means the level, amount or concentration of an active ingredient needed to treat a symptom, disease, disorder, or condition without causing significant negative or adverse side effects.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

The use of peptides as delivery vehicles can be achieved through covalent conjugation or physical complexation of the targeting peptide to its cargo. Covalent conjugation requires expertise in peptide-receptor interactions to optimize the site of drug attachment without affecting drug potency and receptor binding, and the selectivity of the peptide via steric hindrance. A proper bond or linker should not affect the affinity and specificity of the peptide, and should render the drug conjugate stable until it reaches the target where the drug can be released. Physical complexation is readily executed by simply mixing the targeting peptide with the cargo drug, but the formation of noncovalent interactions is dependent on the physiochemical properties of the two interacting components and the formulation. This approach tolerates varying molar mixing ratios, however, it also produces a mixture of nonhomogeneous structures.

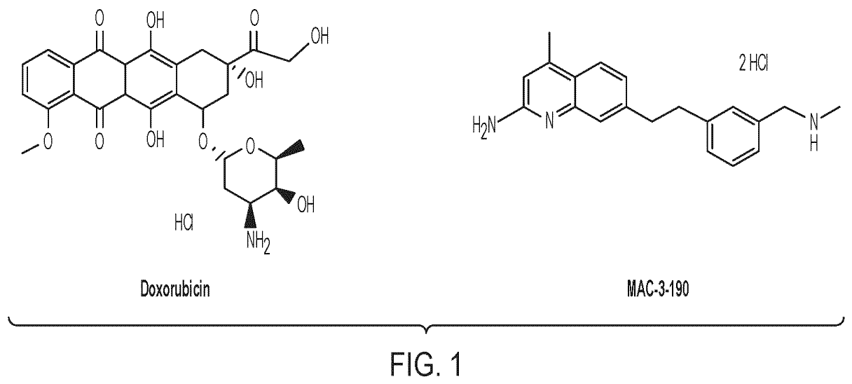

Small molecule drugs possess advantages, including favorable pharmacokinetics, low production costs, and high patient compliance. Our previous studies have shown that neuronal nitric oxide synthase (nNOS) expression levels in patient biopsies significantly correlated with the disease stage and that nNOS inhibitors may be a promising direction for melanoma treatment (Huang et al. J Med Chem 57:686-700, 2014; Yang et al. Antioxid Redox Signal 19:433-447, 2013). We found that several small molecule nNOS inhibitors, such as MAC-3-190 (FIG. 1), exhibit potent and promising anti-melanoma activities. However, nitric oxide (NO) is involved in regulating muscle tone in the sphincter of the lower esophagus, pylorus, sphincter of Oddi, and anus. Gastrointestinal changes were reported in animal studies after nNOS inhibitor treatment, with delayed gastric emptying and colonic transit. In a recent study, antibiotics were found to alter the expression of nNOS in the murine gut, resulting in similar observations along with an increase in the thickness of muscularis externa in the stomach, ileum, and cecum. Enlarged stomachs with hypertrophy of the pyloric sphincter were also observed in transgenic mice with homozygous depletion of the nNOS gene.

In this study, we synthesized a peptide library on a functionalized cellulose membrane to screen for a peptide with specific affinity for melanoma cells and low binding to non-melanoma cells. We identified a 12-mer peptide, KK-11 (VPWXEPAYQRFL), for targeting melanoma and a D-amino acid substituted analogue of KK-11 (VPWxEPAYQrFL) was synthesized to improve its stability in serum. The peptide is specifically taken up by melanoma cells both in vitro and in vivo.

We then determined whether KK-11 improved the anti-melanoma activities of the tested drugs. Two types of anti-cancer drugs were studied, cytotoxic DOX and nNOS inhibitor MAC-3-190. The latter is a targeted therapy for melanoma. Co-administration of KK-11 significantly enhanced the cytotoxicity of DOX in vitro, and the in vivo antitumor activity of MAC-3-190 in a melanoma xenograft mouse model. Thus, peptide KK-11 can be used as a carrier or targeting ligand to improve drug delivery to melanoma cells and enhance anti-melanoma activity.

To develop the disclosed compositions and methods, a peptide library array was designed and screened using a peptide array-whole cell binding assay, which identified KK-11 as a novel human melanoma-targeting peptide. The peptide and its D-amino acid substituted analogue (VPWxEPAYQrFL or D-aa KK-11) were synthesized via a solid-phase strategy. Further studies using FITC-labeled KK-11 demonstrated dose-dependent uptake in human melanoma cells. D-aa KK-11 significantly increased the stability of the peptide, with 45.3% remaining detectable after 24 hours with human serum incubation.

Co-treatment of KK-11 with doxorubicin (DOX) was found to significantly enhance the cytotoxicity of DOX compared to DOX alone or sequential KK-11 and DOX treatment. In vivo and ex vivo imaging revealed that D-aa KK-11 distributed to xenografted A375 melanoma tumors as early as 5 minutes and persisted up to 24 hours post tail vein injection. When co-administered, D-aa KK-11 significantly enhanced the anti-tumor activity of a novel nNOS inhibitor (MAC-3-190) in an A375 human melanoma xenograft mouse model compared to MAC-3-190 treatment alone. No apparent systemic toxicities were observed. Taken together, these results suggest that KK-11 may be a promising human melanoma-targeted delivery vector for anti-melanoma cargo.

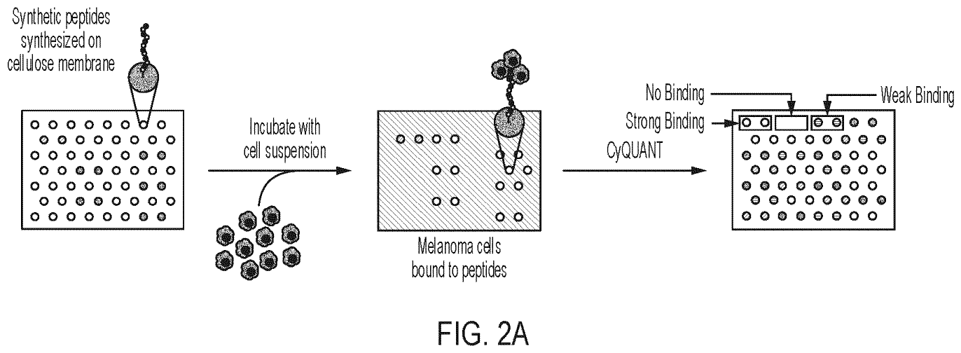

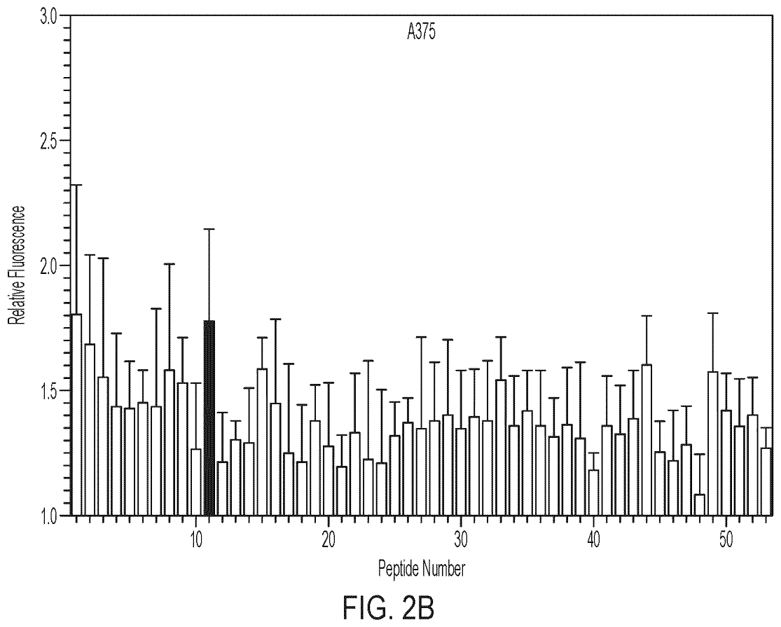

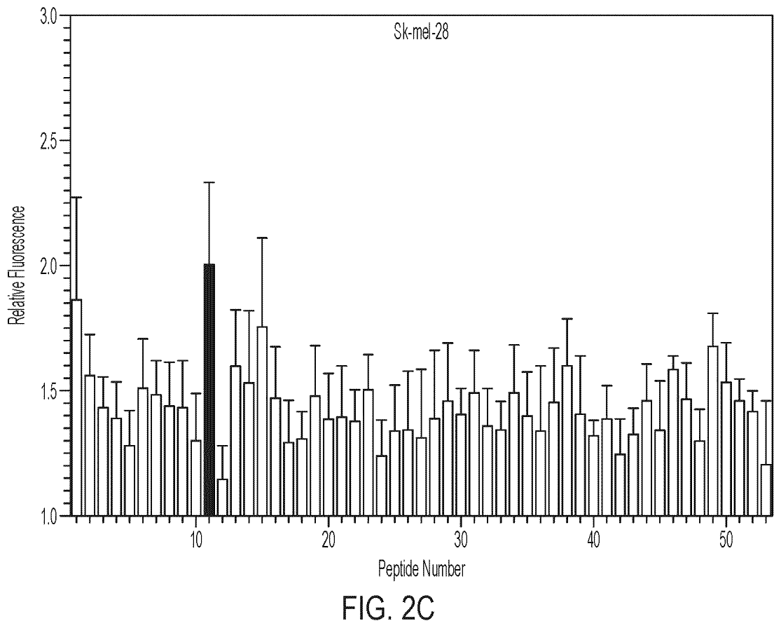

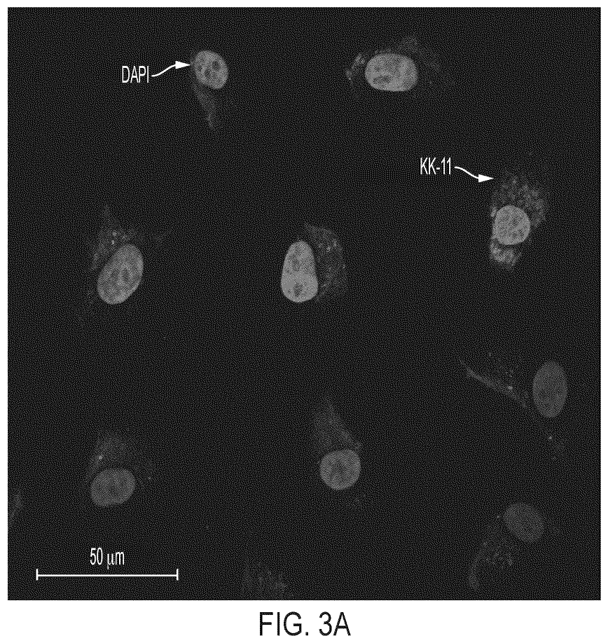

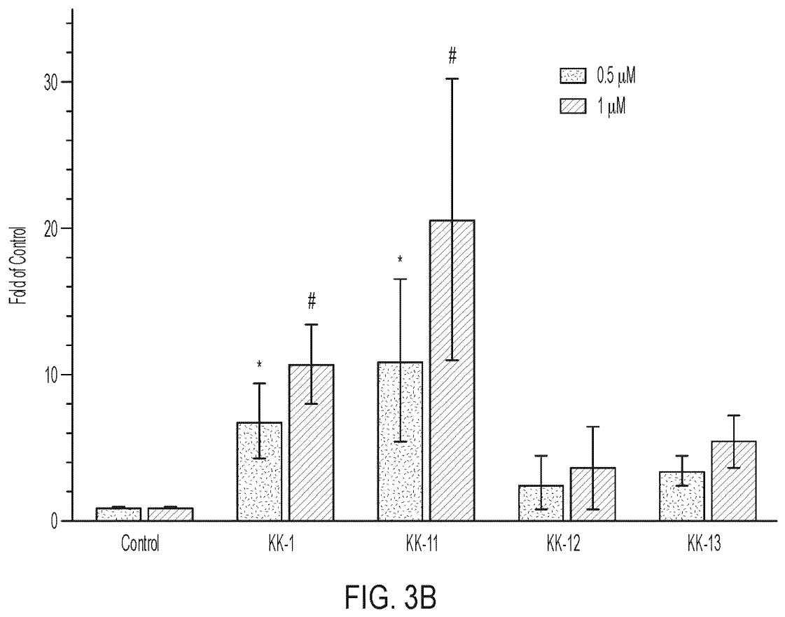

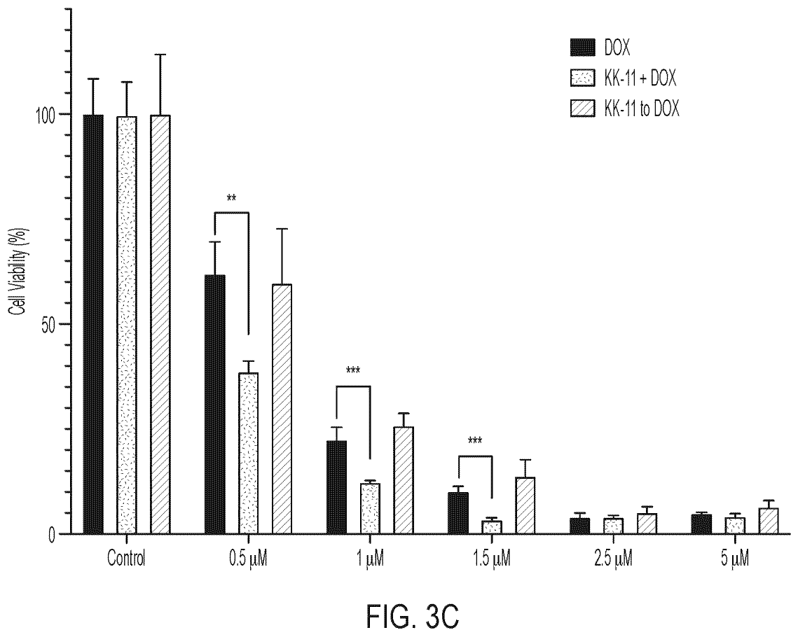

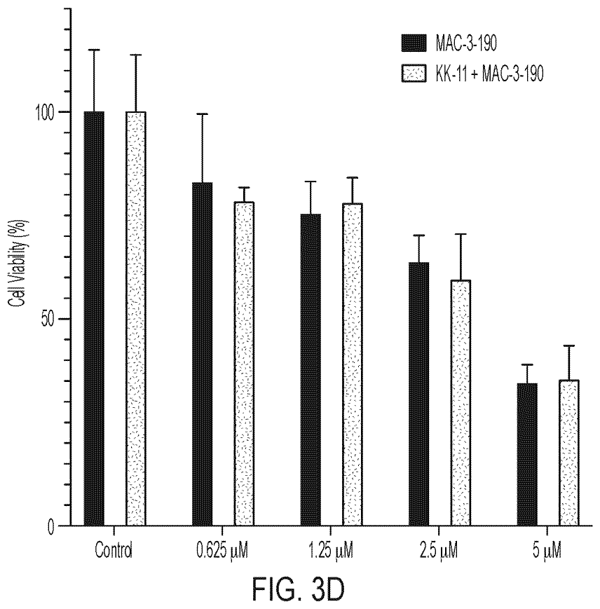

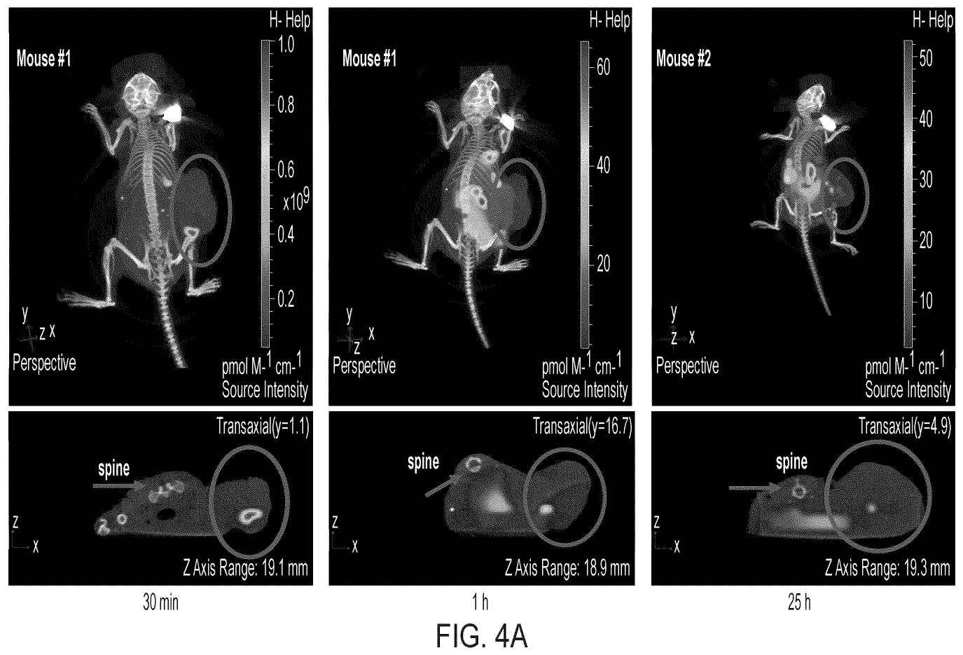

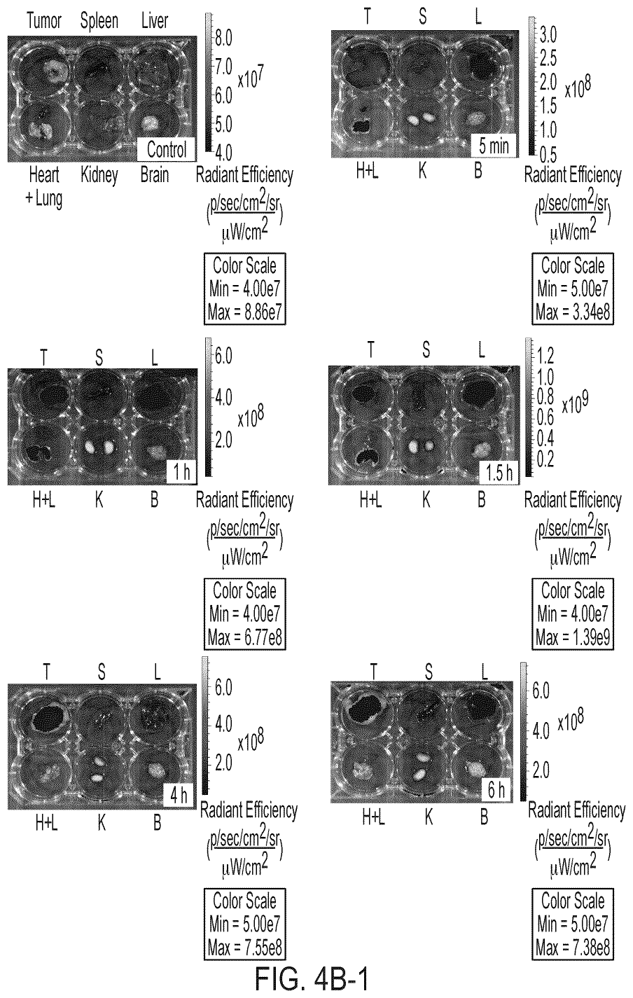

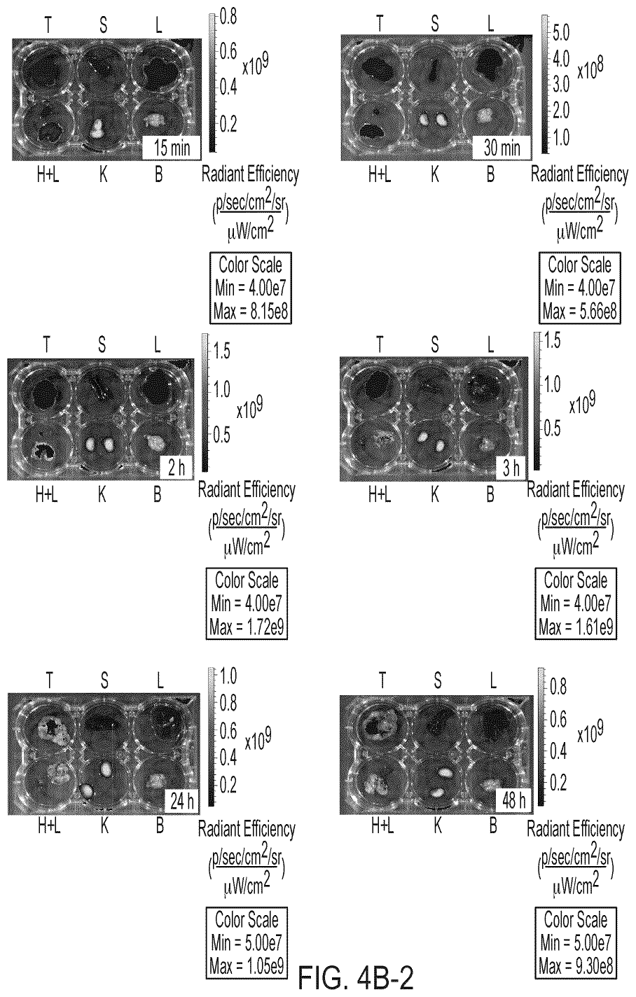

KK-11 specifically binds to wm115 (primary), A375 (metastatic), and Sk-mel-28 (metastatic) melanoma cell lines compared to normal HEK-293 cells (FIG. 2). FITC-labeled KK-11 also demonstrated dose-dependent uptake in melanoma cells, suggesting KK-11 can be a sufficient carrier to improve the drug delivery to melanoma (FIG. 3). In this study, we observed that KK-11 can also bind to human primary glioma U87 cells (data not shown here). Further, structural optimization via D-amino acid substitution significantly improved serum stability. After a single injection, the peptide (VivoTag-KK-11) remains detectable in the tumor xenografts for at least 24 h, as shown in our in vivo and ex vivo biodistribution analysis (FIG. 4). Co-treatment of KK-11 was shown to enhance the cytotoxicity of DOX significantly (FIG. 3C). However, such effects of KK-11 were not evident after sequential treatment. These results indicate that KK-11 serves as a targeted delivery vehicle and forms a noncovalent complex with DOX as the cargo, shuttling it into the cell to enhance DOX cytotoxicity in A375 melanoma cells. Previous studies also showed that co-administration of a tumor-targeting peptide iRGD with chemotherapeutic agents, like doxorubicin, nab-paclitaxel (nanoparticles), or trastuzumab (antibody), enhanced the efficacy of cancer drugs.

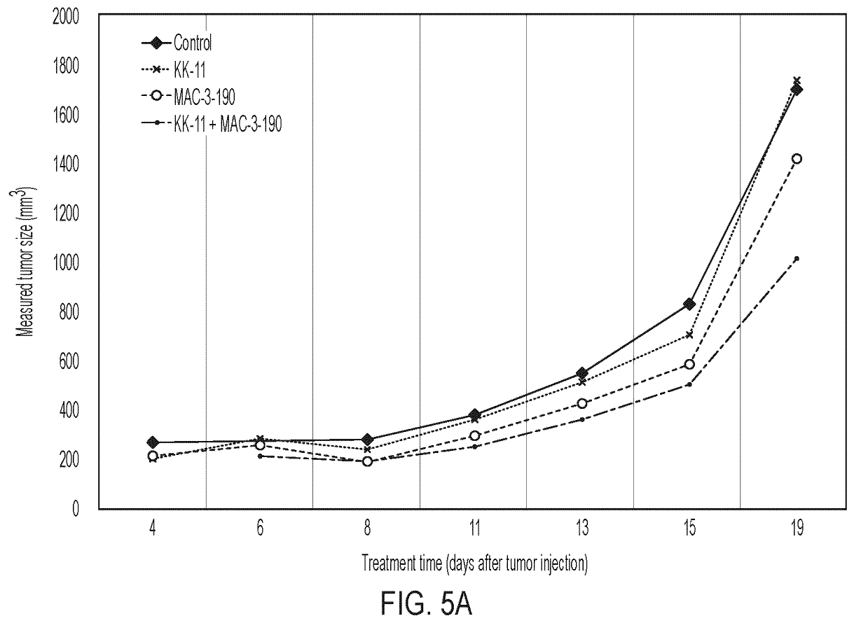

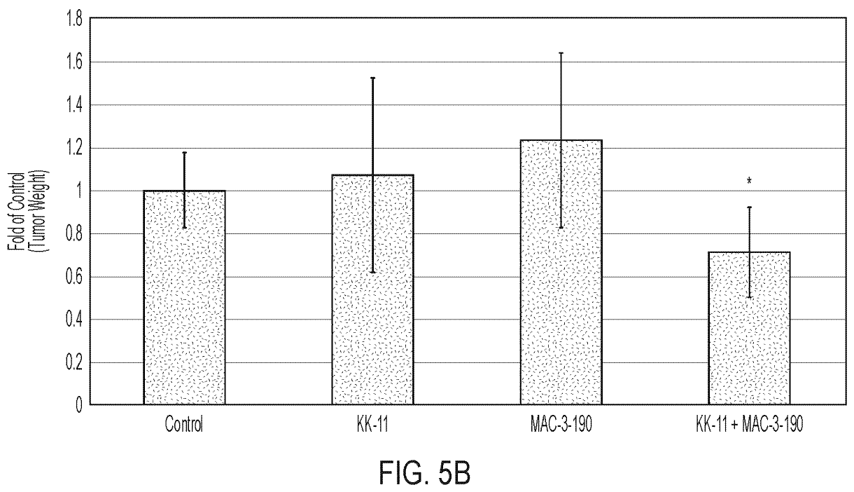

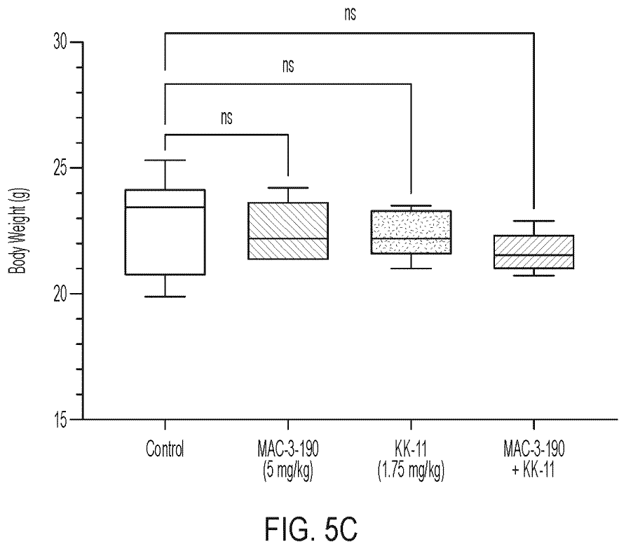

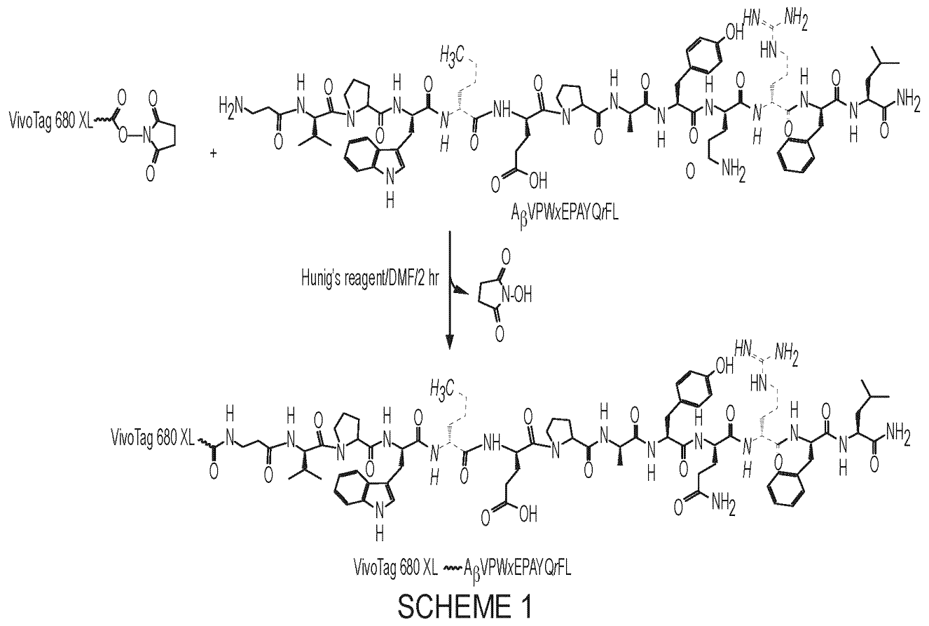

In the current animal study (FIG. 5C), the median body weight in MAC-3-190 treatment group was lower compared to the control group but was not statistically significant (p>0.05). Given the potential gastrointestinal adverse effects associated with nNOS inhibition, our strategy aims to blend targeted therapy (nNOS inhibitors) with targeted delivery by coupling the small molecule with KK-11 to optimize the anti-melanoma treatment. By using this approach, the effective dosage of nNOS inhibitors can be lowered without compromising their anti-tumor activities. In this study, we examined whether KK-11 could enhance the in vivo anti-melanoma activity of a small molecule nNOS inhibitor MAC-3-190 (FIG. 5).

In contrast to the effects observed in A375 cells with doxorubicin treatment (FIG. 3C), co-incubation of KK-11 with MAC-3-190 did not enhance cytotoxicity in vitro (FIG. 3D). However, our in vivo study demonstrated that co-administration of KK-11 with the selective nNOS inhibitor MAC-3-190 significantly reduced tumor growth compared to control and MAC-3-190 alone (FIG. 5A), while the antitumor activity was not observed with low-dose MAC-3-190 treatment alone. Of note, the co-treatment group's body weight was lower compared to that of the groups treated with MAC-3-190 alone or the vehicle control (FIG. 5C). However, such a difference was not statistically significant by ordinary one-way ANOVA analysis. No cytotoxicity of KK-11 alone was observed in our in vitro or in vivo studies.

Methods of Treatment

Aspects of the methods of the present disclosure include, in part, treatment of a mammal. A mammal includes a human, and a human can be a patient. Other aspects of the present disclosure provide, in part, an individual. An individual includes a mammal and a human, and a human can be a patient.

Disclosed herein are methods of treating a cancer by administering a combination of a tumor-targeting peptide and at least one chemotherapeutic agent. In some embodiments, the tumor-targeting peptide and at least one chemotherapeutic agent are administered individually. In some embodiments, the tumor-targeting peptide and chemotherapeutic agent are conjugated.

In some embodiments, the cancer is selected from bladder cancer, bone cancer, brain cancer, breast cancer, colon and rectal cancer, esophageal cancer, gastric cancer, a gynecologic cancer (i.e., cervical cancer, ovarian cancer, endometrial cancer, uterine cancer, vaginal cancer, and vulvar cancer, etc.), head and neck cancer, kidney cancer, leukemia, liver cancer, lung cancer, lymphoma (i.e., Hodgkin's lymphoma, non-Hodgkin's lymphoma, etc.), melanoma, mesothelioma, myeloma, neuroblastoma, pancreatic cancer, prostate cancer, retinoblastoma, rhabdomyosarcoma, skin cancer, testicular cancer, and thyroid cancer.

In some embodiments, the cancer is melanoma.

Administration in methods disclosed herein include a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; buccal, nasal, and/or inhalation administration in any acceptable form; rectal administration in any acceptable form; vaginal administration in any acceptable form; intravascular administration in any acceptable form, such as, e.g., intravenous bolus injection, intravenous infusion, intra-arterial bolus injection, intra-arterial infusion and catheter instillation into the vasculature; peri- and intra-tissue administration in any acceptable form, such as, e.g., intraperitoneal injection, intramuscular injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a stent, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

Compositions disclosed herein can be administered to a mammal using a variety of routes. Routes of administration suitable for treating a cancer as disclosed herein include both local and systemic administration. Local administration results in significantly more delivery of a combination to a specific location as compared to the entire body of the mammal, whereas, systemic administration results in delivery of a combination to essentially the entire body of the individual. Routes of administration suitable for or treating a cancer as disclosed herein also include both central and peripheral administration. Central administration results in delivery of a combination to essentially the central nervous system of the individual and includes, e.g., nasal administration, intrathecal administration, epidural administration as well as a cranial injection or implant. Peripheral administration results in delivery of a compound or a combination to essentially any area of an individual outside of the central nervous system and encompasses any route of administration other than direct administration to the spine or brain.

The actual route of administration of a compound or a combination disclosed herein used can be determined by a person of ordinary skill in the art by taking into account factors, including, without limitation, the type of cancer, the location of the cancer, the severity of the cancer, the duration of treatment desired, the degree of relief desired, the duration of relief desired, the particular compound or combination used, the rate of excretion of the compound or combination used, the pharmacodynamics of the compound or combination used, the nature of the other compounds to be included in the combination, the particular route of administration, the particular characteristics, history and risk factors of the individual, such as, e.g., age, weight, general health and the like, the response of the individual to the treatment, or any combination thereof. An effective dosage amount of a compound or a combination disclosed herein can thus readily be determined by the person of ordinary skill in the art considering all criteria and utilizing his best judgment on the individual's behalf.

In an embodiment, a combination disclosed herein is administered systemically to a mammal. In another embodiment, a combination disclosed herein is administered locally to a mammal. In an aspect of this embodiment, a combination disclosed herein is administered to a site of a cancer in a mammal.

In embodiments, a tumor-targeting peptide disclosed herein and at least one chemotherapeutic agent do not need to be administered by the same route or on the same administration schedule.

Dosing can be single dosage or cumulative (serial dosing), and can be readily determined by one skilled in the art. For instance, treatment of a cancer may comprise a one-time administration of an effective dose of a combination disclosed herein. As a non-limiting example, an effective dose of a combination disclosed herein can be administered once to a mammal, e.g., as a single injection or deposition at or near the site of a tumor or a single oral administration of the combination. More advantageously, treatment of a cancer may comprise multiple administrations of an effective dose of a combination disclosed herein carried out over a range of time periods, such as, e.g., daily, once every few days, weekly, monthly or yearly. As a non-limiting example, a combination disclosed herein can be administered once or twice weekly to a mammal. The timing of administration can vary from mammal to mammal, depending upon such factors as the severity of a mammal's symptoms.

For example, an effective dose of a combination disclosed herein can be administered to a mammal once a month for an indefinite period of time, or until the mammal no longer requires therapy. A person of ordinary skill in the art will recognize that the condition of the mammal can be monitored throughout the course of treatment and that the effective amount of a combination disclosed herein that is administered can be adjusted accordingly. Additionally, each element of the combination, for example the tumor-targeting agent and at least one chemotherapeutic agent, can be administered by different routes and on different schedules and are optionally administered individually, although both compositions (the tumor-targeting agent and chemotherapeutic agent) are administered to the individual such that plasma levels of both compounds are detectable at the same time.

A combination disclosed herein as disclosed herein can also be administered to a mammal in combination with other therapeutic compounds to increase the overall therapeutic effect of the treatment. The use of multiple compounds to treat an indication can increase the beneficial effects while reducing the presence of side effects.

Compositions

Disclosed compositions can comprise a combination of a tumor-targeting peptide and a chemotherapeutic agent. The combination can comprise a combination formulation or the components can be combined at the time of administration.

In some embodiments, the chemotherapeutic agent comprises an alkylating agent (i.e, cyclophosphamide, mechlorethamine, chlorambucil, melphalan, dacarbazine, nitrosoureas, temozolomide), an anthracycline (i.e., daunorubicin, doxorubicin, epirubicin, idarubicin, mitoxantrone, valrubicin), a taxane (i.e., paclitaxel, docetaxel, abraxane, taxotere), epithilone, a histone deacetylase inhibitor (i.e., vorinostat, romidepsin), a topoisomerase inhibitor (i.e., irinotecan, topotecan, etoposide, teniposide, tafluposide), a kinase inhibitor (i.e., bortexomib, erlotinib, gefitinib, imatinib, vemurafenib, vismodegib), a nucleoside analog (i.e., azacitidine, azathioprine, capecitabine, cytarabine, doxifluridine, fluorouracil, gemcitabine, hydroxyurea, mercaptopurine, methotrexate, tioguanine), a peptide antibiotic (i.e., bleomycin, actinomycin), a platinum-based agent (i.e., carboplatin, cisplatin, oxaliplatin), a retinoid (i.e., tretinoin, alitretinoin, bexarotene), a vinca alkaloid (i.e., vinblastine, vincristine, vindesine, vinorelbine), or a neuronal nitric oxide synthase inhibitor (i.e., MAC-3-190, HH044).

A combination of a tumor-targeting peptide disclosed herein and a chemotherapeutic agent, is generally administered to an individual as a pharmaceutical composition. Pharmaceutical compositions may be prepared by combining a therapeutically effective amount of at least one tumor-targeting peptide and at least one chemotherapeutic agent, as an active ingredient, with conventional acceptable pharmaceutical excipients, and by preparation of unit dosage forms suitable for therapeutic use. As used herein, the term “pharmaceutical composition” refers to a therapeutically effective concentration of an active compound, such as, e.g., any of the compounds disclosed herein. Preferably, the pharmaceutical composition does not produce an adverse, allergic, or other untoward or unwanted reaction when administered to an individual. A pharmaceutical composition disclosed herein is useful for medical and veterinary applications. A pharmaceutical composition may be administered to an individual alone, or in combination with other supplementary active compounds, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir, or any other dosage form suitable for administration.

A pharmaceutical composition produced using the methods disclosed herein may be a liquid formulation, semi-solid formulation, or a solid formulation. A formulation disclosed herein can be produced in a manner to form one phase, such as, e.g., an oil or a solid. Alternatively, a formulation disclosed herein can be produced in a manner to form two phase, such as, e.g., an emulsion. A pharmaceutical composition disclosed herein intended for such administration may be prepared according to any method known to the art for the manufacture of pharmaceutical compositions.

Liquid formulations suitable for parenteral injection or for nasal sprays may comprise physiologically acceptable sterile aqueous or nonaqueous solutions, dispersions, suspensions or emulsions and sterile powders for reconstitution into sterile injectable solutions or dispersions. Formulations suitable for nasal administration may comprise physiologically acceptable sterile aqueous or nonaqueous solutions, dispersions, suspensions or emulsions. Examples of suitable aqueous and nonaqueous carriers, diluents, solvents or vehicles include water, ethanol, polyols (propylene glycol, polyethyleneglycol (PEG), glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Pharmaceutical formulations suitable for administration by inhalation include fine particle dusts or mists, which may be generated by means of various types of metered, dose pressurized aerosols, nebulizers, or insufflators.

Semi-solid formulations suitable for topical administration include, without limitation, ointments, creams, salves, and gels. In such solid formulations, the active compound may be admixed with at least one inert customary excipient (or carrier) such as, a lipid and/or polyethylene glycol.

Solid formulations suitable for oral administration include capsules, tablets, pills, powders and granules. In such solid formulations, the active compound may be admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example, paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

A pharmaceutical composition disclosed herein can optionally include a pharmaceutically acceptable carrier that facilitates processing of an active compound into pharmaceutically acceptable compositions. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003). These protocols are routine and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

The combination of a tumor-targeting peptide and a chemotherapeutic agent may also be incorporated, together or separately, into a drug delivery platform in order to achieve a controlled release profile over time. Such a drug delivery platform comprises the combination disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a drug delivery platform are described in, e.g., U.S. Pat. Nos. 4,756,911; 5,378,475; 7,048,946; U.S. Patent Publication 2005/0181017; U.S. Patent Publication 2005/0244464; U.S. Patent Publication 2011/0008437; each of which is incorporated by reference in its entirety.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

One of ordinary skill in the art appreciates that the selection of a suitable polymer for forming a suitable disclosed drug delivery platform depends on several factors. The more relevant factors in the selection of the appropriate polymer(s), include, without limitation, compatibility of polymer with drug, desired release kinetics of drug, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, dictate the in vitro and in vivo behavior of the polymer include the chemical composition, spatial distribution of the constituents, the molecular weight of the polymer and the degree of crystallinity.

A drug delivery platform includes both a sustained release drug delivery platform and an extended release drug delivery platform. As used herein, the term “sustained release” refers to the release of a compound or combination disclosed herein over a period of about seven days or more. As used herein, the term “extended release” refers to the release of a compound or combination disclosed herein over a period of time of less than about seven days.

In aspects of this embodiment, a sustained release drug delivery platform releases a one or both of a tumor-targeting peptide and at least one chemotherapeutic agent with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a combination disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a drug delivery platform releases a combination disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases a combination disclosed herein with substantially first order release kinetics over a period of, e.g., at most 1 day after administration, at most 2 days after administration, at most 3 days after administration, at most 4 days after administration, at most 5 days after administration, or at most 6 days after administration.

Aspects of the present specification may also be described as follows:

### Examples

### Example 1. A Small Peptide Increases Drug Delivery in Human Melanoma Cells

Materials and Methods

Peptide array syntheses were conducted using an automated spot synthesis system (ResPep SL) as before (Intavis AG, Germany). Individual peptide synthesis was performed on an automated peptide synthesizer (Tribute) from Protein Technologies (Tucson, AZ). Purification of peptides used the Prominence-i HPLC System (Shimadzu, Kyoto, Japan). The ChemiDoc™ XRS+system (Bio-Rad, CA) was used to record fluorescence intensity on the membranes.

Rink amide resin, L- and D-protected amino acid building blocks, and chemical reagents were purchased from AAPPTec. All solvents used for HPLC were obtained from Sigma-Aldrich, USA and used without further purification. VivoTag 680 XL (665/688 nm Excitation/Emission wavelength) was purchased from PerkinElmer. The targeted peptides were purified by RP-HPLC with a Shimadzu, C₁₈ (19×250 mm) column, and the purity was confirmed by analytical RP-HPLC using a mobile phase composed of eluent A (99.9% v/v H₂O and 0.1% v/v TFA) and eluent B (99.9% v/v CH3CN and 0.1% v/vTFA). Mass spectra were recorded with a Bruker Daltonics Autoflex MALDI-TOF using α-cyano-4-hydroxycinnamic acid (CHCA) as the matrix. ¹H and ¹³C NMR spectra were obtained on a Bruker Avance III HD™ 400 NMR spectrometer (internal standard TMS), using deuterated methanol as solvent.

A Milli-Q system was used for ultrapure water. Sterile water was from Millipore Sigma. DMEM, Step/Pen, Hank's Balanced Salt Solution (HBSS), 0.05% trypsin/EDTA, and horse serum were obtained from Gibco and Hyclone. All solvents and chemicals, including triisopropylsilane (TIPS), dichloromethane (DCM), dimethylformamide (DMF), N-methylmorpholine (NMM), diethyl ether, ethanol, acetonitrile, trifluoroacetic acid (TFA), diisopropylcarbodiimide (DIC), ethyl cyanohydroxyiminoacetate (Oxyma pure), O-(1H-6-chlorobenzotriazole-1-yl)-1,1,3,3-tetramethyluronium hexafluorophosphate (HCTU), and N, N-diisopropylethylamine (DIPEA) were obtained from Sigma-Aldrich. Methanol ACS was from EMD Millipore. 20% piperidine was purchased from Protein Technology. CyQUANT and FITC dyes were obtained from Invitrogen (Eugene, OR). Derivatized cellulose membranes was from Intavis (AG, Germany). Human serum was purchased from Sigma-Aldrich.

Cell lines. Four human melanoma cell lines carrying distinct genomic mutations (A375: BRAF^{V600E}/PTEN^{WT}/CKIT^{WT}; SK-Mel-28: BRAF^{V600E}/PTEN^{T167A}/CKIT^{WT}; wm115: BRAF^{V600E}/PTEN^{WT}/CKIT^{WT}; and WM3211: BRAF^{WT}/PTEN^{WT}/CKIT^{L576P}) were used for the study [60,61]. A375, wm115, Sk-mel-28, and HEK-293 were purchased from American Type Culture Collection (ATCC; Manassas, VA), and wm3211 was obtained from Rockland Immunochemicals (Limerick, PA). Cell lines were cultured in Dulbecco's Modified Eagle's Medium (DMEM, #11995073; Gibco, Waltham, MA) (A375) or Eagle's Minimum Essential Medium (EMEM) (wm115, SK-Mel-28, HEK-293) with 10% fetal bovine serum (FBS, #26140079; Gibco), or Tumor Specialized Media with 2% FBS (WM3211). Cell culture media were supplemented with 10% FBS (Corning). Cells were cultured in a humidified atmosphere in a 5% CO₂ incubator maintained at 37° C.

Peptide array synthesis. Fifty-three peptide sequences were synthesized in duplicate on derivatized cellulose membrane (Intavis, Germany) using a ResPep SL Autospot robot. The detailed method was explained previously (Ahmed et al. Anal Chem. 82:7533-7541, 2010; Hossein-Nejad-Ariani et al. Sci Rep. 9:2723, 2019). Briefly, the sequences of peptides were entered in the robot software, and 384 spot synthesis mode was selected for this library. First, the membrane was dipped in DMF for an hour followed by transfer and support to the holder of the robot on filter paper. Then the membrane was washed with ethanol and subjected to a vacuum to remove any bubbles under the membrane. The Fmoc strategy was used for peptide synthesis. DIC and Oxyma pure were used as coupling reagent and racemization suppressor, respectively. After each coupling, capping of unreacted amino acids was done using acetic anhydride (3%), followed by 20% piperidine for Fmoc deprotection. The C-terminal end of all peptides were bound to the membrane. Following the synthesis, the membrane was dipped in deprotection solution containing 15 mL TFA, 15 mL DCM, 900 μL TIPS, and 600 μL water, and kept for 3 h at room temperature. Next, membranes were thoroughly washed with DCM, DMF, and ethanol. Finally, they were air dried and kept in a sealed bag at −20° C. until use.

Peptide array cell binding assay. The sequential steps of the assay are as described in a previous publication (Ahmed et al. 2010; Soudy et al. J Med Chem. 54:7523-7534, 2011). The membrane was dipped in ethanol for 30 sec to remove any precipitation of hydrophobic peptides. Then the membrane was dipped in sterile PBS at pH 7.4 for 30 min. The cells (75×10³ cells/mL) were seeded directly on the membrane in a sterile plate and incubated at 37° C. for 4 h. After washing off the unbound cells with sterile PBS, pH 7.4, the membrane was frozen at −80° C. for 2 h. Subsequently, it was defrosted at room temperature and incubated in CyQUANT solution at 37° C. for 30 min following the manufacturer's protocol. The membrane was then washed with sterile PBS and air-dried.

Membranes were scanned using the ChemiDoc™ XRS+system (Bio-Rad) to analyze the fluorescence intensity of each spot. The setting was adjusted based on the excitation and emission wavelength of the CyQUANT dye at 465 nm and 535 nm, respectively. The fluorescence intensity (FI) was normalized by the FI of non-cancerous HEK-293 cells. The relative fluorescence densities were used for identifying peptides that exhibit high binding affinity to melanoma cells.

FITC labeled-peptide synthesis and purification. Four peptides, KK-1 KK-11, KK-12, and KK-13 were selected for uptake studies in human melanoma cells. These four peptides were synthesized based on Fmoc solid-phase peptide synthesis (Fmoc-SPPS) on preloaded Fmoc-Leu/Ala Wang resin (0.1 mmol scale) using an automated peptide synthesizer (Tribute, Protein Technology) and following the procedure reported previously (Raghuwanshi et al. J Med Chem 60:4893-4903, 2017; Soudy et al. J Med Chem. 56:7564-7573, 2013). Preloaded Fmoc leucine/alanine Wang resin (145/227 mg, 0.1 mmol) was added to the glass reaction vessel (RV). Resin swelling was done automatically with nitrogen blowing and mechanical shaking in DMF for 30 min. All amino acids were coupled in sequence using HCTU (2.5 equiv) and NMM (1.2 equiv) for each coupling and was mixed with amino acid (3 equiv) in 3 mL of DMF. Fmoc was removed with 20% piperidine in DMF automatically. β-Alanine was added at the N-terminus of the sequence as a spacer for FITC coupling. In the final step of the automated synthesis, an extra DCM washing was added to prepare resins for peptide cleavage. FITC (0.3 mmol) was mixed in 5 mL DMF with DIPEA (Hunig's reagent) (0.15 mmol) and then incubated with the resin in the dark for 20 h. The successful conjugation was confirmed using MALDI-TOF and RP-HPLC. Cleavage of peptides was done manually. The cleavage cocktail was 95% TFA (9.50 mL), 2.5% TIPS (250 μL), and 2.5% ultra-pure water (250 μL). The peptides were precipitated using cold diethyl ether (20 mL). The purification was done using a semi-preparative RP-HPLC with a C18 Vydac column. The purity of the FITC-peptides was determined from analytical RP-HPLC chromatograms (AUC) and was found to be >95%. All peptides were characterized using MALDI-TOF mass spectrometry. The concentrations of peptides were obtained using the extinction coefficient of FITC. The absorption was read using a Shimadzu Nanodrop, and the concentration was calculated using the Beer-Lambert equation.

Synthesis of VivoTaq-KK-11 peptide. For the synthesis of fluorescent VivoTag 680 XL labeled D-amino acid analogue of KK-11 (VivoTag-KK-11), first, the linear peptide (D-amino acid analog of KK-11, D-aa KK-11) was synthesized following Fmoc SPPS on Rink amide resin (526 mg, 0.30 mmol, 0.57 mmol/g). After the synthesis, the crude targeted peptide was subjected to RP-HPLC for purification. The pure fraction was concentrated and subsequent freeze-drying to afford pure powdered peptide A_{β}VPWxEPAYQrFL. The purity of the peptide was confirmed by analytical RP-HPLC and the molecular weight by MALDI-TOF (m/z), [C₇₈H₁₁₃N₁₉O₁₇]: Calcd [M+H]⁺, 1588.9; Found [M+H]⁺, 1588.5. Next, in an amber vial, D-aa KK-11 (2.00 mg, 0.0012 mmol) was dissolved in DMF (0.30 mL) followed by adding a solution of VivoTag 680 XL (0.57 mg, 0.0003 mmol, 0.3 equiv) in DMF (0.2 mL). The mixture was treated with Hunig's reagent (3.12 μL, 0.018 mmol, 15 equiv) followed by stirring at room temperature for 2 h. After completion of the reaction, the solution was concentrated and added to cold ether, affording the green crude VivoTag-KK-11, which was subjected to analytical RP-HPLC for purification (Rₜ=45 min). MALDI-TOF (m/z), [C₇₅H₁₀₈N₁₈O₁₆]: Calcd [M+H]⁺, 2823.2 Da; Found [M+H]⁺, 2823.4 Da.

Fluorescence microscopy and imaging. A375 melanoma cells were plated on coverslips and allowed to adhere overnight to 75% confluence, then incubated with 0.5 μM of FITC-KK-11 for 30 min at room temperature. After treatment, cells were washed three times in 1×PBS then fixed with 4% formaldehyde in 1×PBS for 15 min at room temperature. Fixed cells were then washed three times in 1×PBS for 5 min each followed by curing in the dark with ProLong™ Gold Antifade Mountant with DAPI (P36935, Life Technologies Corporation, Eugene, OR) for 1 h. Slides were visualized and recorded using the Nikon Eclipse Ti2-E confocal microscopy system (Nikon, Melville, NY) using green and blue filters with 60× magnification.

In vitro cellular uptake analysis. The peptide (FITC-labeled) concentration was determined using a Shimadzu BioSpec-nano Micro-volume UV-Vis spectrophotometer (Shimadzu). Human melanoma cells (wm3211, Sk-mel-28, and A375) were incubated with FITC or FITC-labeled peptides at final concentrations of 0.5 μM and 1 μM for 2 h. After thorough washing with cold 1×PBS, the melanoma cells in a single cell suspension were collected for flow cytometry analysis (BD FACSVerse, BD Biosciences, CA). The mean fluorescence density of 10,000 cells was analyzed and compared to that of control cells.

Serum stability. Human serum (250 μL) was thawed to room temperature and was added to DMEM media (650 μL). The mixture was kept in a 37° C. incubator to mimic human body temperature for 15 min. The peptide (100 μL, 1 mM) was dissolved (dispersed) in sterile water. The peptide solution was added to the pre-warmed human serum and DMEM mixture. The mixture was incubated at 37° C. At different time points (0, 0.5, 1, 5, and 24 h) aliquots (100 μL) were removed and added to methanol (200 μL). Each time point sample was kept at 4° C. for 5 min and then was centrifuged at 500×g for 15 min to separate serum proteins. The supernatant was injected into the RP-HPLC, and the major peaks were collected. The peak's mass was determined using MALDI-TOF mass analysis. The serum stabilities of the D-aa KK-11 were determined by comparing the HPLC peaks (AUC) for the intact peptides at different time points.

In vitro cytotoxicity detected by MTT colorimetric assay. Cells were seeded in a 96-well plate and allowed to adhere overnight prior to adding the compound. After 72 h of treatment in serum-free media, an MTT solution was added to each well to give a final concentration of 0.5 mg/mL and was incubated for 3 h. Formed crystals were solubilized, and the absorbance was measured at 595 nm.

Ex vivo and in vivo imaging. The study was carried out in compliance with Animal Research: Reporting of In Vivo Experiments (ARRIVE) guidelines. All the animal procedures were approved by the Institutional Animal Care and Use Committee (IACUC) at Chapman University and conducted in compliance with the policies of Chapman University, and federal, state, and local animal welfare authorities. Male and female athymic nude mice (4-6 weeks old) were kept (5 in each cage) in a pathogen-free environment. Approximately 10⁶ human melanoma A375 cells (0.1 mL in normal saline), mixed with 0.1 mL cold Matrigel (#354248; Corning, Corning, NY), were injected subcutaneously in the flank. The tumor size was measured with a caliper, and body weight was recorded once a week. When the tumor size reached approximately 800-1000 mm³ as calculated using the following formula: [Length×(Width²)]/2 (in mm), mice were processed for in vivo or ex vivo imaging.

For in vivo imaging, normal saline or VivoTag-KK-11 at a dose of 8 μg per mouse was injected into the mice via the tail vein (n=2 for each time point). The animals were then scanned at different time intervals (0.5, 2, 6, and 24 h) using an IVIS Spectrum in vivo imaging system (PerkinElmer, Waltham, MA). The mice were imaged for 0.5 sec, 10 bin, level B at an emission wavelength of 688±5 nm. Imaging was limited to no more than once a day and was conducted under continuously maintained isoflurane. Mice were maintained on alfalfa-free diets to minimize the background fluorescence. All the study animals survived through the in vivo imaging procedure.

For ex vivo imaging at defined time intervals (5, 15, 30 min, followed by 1, 1.5, 2, 3, 4, 6, 24, and 48 h) following fluorescence conjugate injection, animals were euthanized via carbon dioxide inhalation (n=2 for each time point); tumor, liver, spleen, heart, lungs, brain, and kidneys were collected and briefly rinsed with saline. The organs were then imaged for 0.5 sec, 10 bin, level B at an emission wavelength of 688±5 nm using an IVIS Spectrum imaging system.

In vivo xenograft melanoma mouse model. Nude mice (Nu/Nu) were purchased from Charles River (Wilmington, MA) and were housed and maintained in the Chapman University vivarium under pathogen-free conditions. A375 cells were suspended in cold Matrigel and injected subcutaneously into the flank of each mouse (1×10⁶ cells per mouse) to establish tumors. The mice were treated by tail vein injections of 1×PBS, D-aa KK-11 alone (1.75 mg/kg), MAC-3-190 (5 mg/kg) alone, or D-aa KK-11 and MAC-3-190 co-treatment for 21 days. For the co-treatment, the solution of D-aa KK-11 and MAC-3-190 (1:11 molar ratio) was mixed gently and incubated at room temperature for 30 min before injection via tail vein. The growth of the tumors was monitored three times a week and measured using digital Vernier calipers. The tumor volume (mm³) was calculated as [Length×(Width²)]/2 (in mm). The mice were sacrificed after 21 days per IACUC policy when the control group's average tumor size reached 2,000 mm³. Tumor xenografts were removed and weighed.

Statistical analyses. All in vitro experiments were repeated at least twice and performed in at least two different human melanoma cell lines. Data shown are means±SD from a representative of at least two independent experiments. Statistical analysis was performed using the student t-test, and a p-value of less than 0.05 was considered statistically significant.

Peptide Library Synthesis and Screeninq

A library of 53 peptides was synthesized in an array format on a cellulose membrane using SPOT synthesis (Table 1). The peptide library was designed starting with five lead cancer cell targeting peptides, Arg peptide (1, Table 1), p160 analogue (11), RGD (16), GE11 (20), and LSD (37). Conservative substitutions, deletion from the N- or C-terminus, alanine scan, and scrambling of amino acids were used to design the analogues for the library. The peptides were screened for specific binding to three human melanoma cell lines. The cells were incubated with the cellulose membranes with conjugated peptides followed by incubation with CyQUANT fluorescence dye. A brief schematic for the peptide array-whole cell binding assay for screening peptides with high affinity for melanoma cells is shown in FIG. 2A. The relative cell adhesion capacities of the peptides to A375 (FIG. 2B), Sk-mel-28 (FIG. 2C), and wm115 were estimated based on the fluorescence of the bound melanoma cells compared to normal human HEK-293 cells. Two peptides, 1 and 11, showed high specificity for melanoma cells as evidenced by the highest fluorescence ratio for melanoma to non-melanoma cell binding (Table 1; flouorescence ratio is the average ratio of fluorescent intensity of melanoma cancer calles (A373/Sk-mel-28, and wm115) bound to a peptide divided by that of non-melanoma HEK-293 cells bound to the same peptide).

TABLE 1
Peptide library screening for
melanoma cell binding.

| Peptide No./ | | Fluorescence |
|---|---|---|
| SEQ. ID No: | Amino Acid Sequence | Ratio |
| 1 | GRRPRPRPRP | 1.82 |
| 2 | GRRPRPRPRPW | 1.60 |
| 3 | GRRPRPRPRPWW | 1.50 |
| 4 | GRRPRPRPRPWWW | 1.46 |
| 5 | GRRPRPRPRPWWWW | 1.43 |
| 6 | GARPRPRPRP | 1.37 |
| 7 | GRAPRPRPRP | 1.47 |
| 8 | GRRPAPRPRP | 1.57 |
| 9 | GRRPRPAPRP | 1.51 |
| 10 | GRRPRPRPAP | 1.31 |
| 11 | VPWXEPAYQRFL | 1.80 |
| 12 | WXEAAYQRFL | 1.31 |
| 13 | EPAAYQRFTA | 1.39 |
| 14 | RVPWLEAPYA | 1.42 |
| 15 | FVPWLEAPYA | 1.61 |
| 16 | RGDAAYQRFL | 1.47 |
| 17 | RGEPAYQRFL | 1.30 |
| 18 | RGEPAYQGRFL | 1.24 |
| 19 | RGDPAYQGRFL | 1.35 |
| 20 | YHWYGTPQNVI | 1.20 |
| 21 | WQTNYIHPYVYG | 1.21 |
| 22 | YGPWYNHYITQV | 1.28 |
| 23 | AHWYGYTPQNVI | 1.31 |
| 24 | YAWYGYTPQNVI | 1.15 |
| 25 | YHAYGYTPQNVI | 1.28 |
| 26 | YHWAGYTPQNVI | 1.29 |
| 27 | YHWYGATPQNVI | 1.19 |
| 28 | YHWYGYAPQNVI | 1.26 |
| 29 | YHWYGYTAQNVI | 1.32 |
| 30 | YHWYGYTPANVI | 1.30 |
| 31 | YHWYGYTPQAVI | 1.33 |
| 32 | YHWYGYTPQNAI | 1.29 |
| 33 | YHWYGYTPQNVA | 1.35 |
| 34 | YHWYGYTPENVI | 1.36 |
| 35 | YHWYGYTPQDVI | 1.35 |
| 36 | YHWYGYTPQKVI | 1.23 |
| 37 | CLSDGKRKC | 1.29 |
| 38 | AGRKLDSKA | 1.39 |
| 39 | ADRSKGKLA | 1.27 |
| 40 | ALSDGKRKA | 1.23 |
| 41 | ALSGKRKC | 1.32 |
| 42 | CASDGKRKC | 1.25 |
| 43 | CLADGKRKC | 1.26 |
| 44 | CLSAGKRKC | 1.44 |
| 45 | CLSDAKRKC | 1.22 |
| 46 | CLSDGARKC | 1.35 |
| 47 | CLSDGKAKC | 1.27 |
| 48 | CLSDGKRAC | 1.12 |
| 49 | CLSDGKRKA | 1.57 |
| 50 | CLSEGKRKC | 1.38 |
| 51 | CLSDGRRKC | 1.32 |
| 52 | CLSDGKRRC | 1.27 |
| 53 | WLSDGKRKC | 1.12 |

Based on our previous studies and published literature, a library containing 53 peptides in duplicates was designed and synthesized on cellulose membranes using an automated spot synthesis ResPep SL apparatus (Intavis AG, Germany). The lead peptides for the library design were Arg peptide (1), p160 analogue (11), RGD (16), GE11 (20), and LSD (37). X stands for norleucine.

TABLE 2
Sequence and MALDI-TOF mass analysis of different KK-11
peptides used in the study.

| Peptide No./ | Amino Acid | | [M + H]⁺ | |
|---|---|---|---|---|
| SEQ. ID No: | Sequence | Label | Calculated | Found |
| 54 | VPWXEPAYQRFL | KK-11 | 1517.8 | 1518.1 |
| 55 | VPWxEPAYQrFL | D-aa KK-11 | 1517.8 | 1517.6 |
| 56 | FITC-A_{β}- | FITC-KK-11 | 1979.2 | 1979.1 |
| | VPWXEPAYQRFL | | | |
| 57 | VivoTag- A_{β}- | VivoTag-KK-11 | 2823.2 | 2823.4 |
| | VPWxEPAYQrFL | | | |

Synthesis of Soluble Fluorescently Labeled Peptides

Soluble peptides labeled with fluorescent FITC were synthesized to evaluate uptake in melanoma cells. FITC was attached to the N-terminus of the peptides, and β-alanine was used as a spacer between the peptide and FITC. Based on the binding studies, four peptides, called KK-1, KK-11, KK-12, and KK-13, were synthesized (Table 1). Peptides 12 and 13 were used as control sequences. The peptides were synthesized using solid phase methodology. FITC labeled peptides were purified using RP-HPLC (purity 95%) and characterized using MALDI-TOF mass spectrometry. Pure peptides were dried and stored at −20° C. until use.

For in vivo studies, peptide KK-11 was labeled with a fluorescent Vivotag 680 XL in the N-terminus (Scheme 1). In addition, two amino acids in the sequence (norleucine and arginine) were substituted with D-amino acids to increase proteolytic stability. These amino acids were identified as proteolytically labile sites. The D-aa KK-11 peptide was assembled on acid-labile Rink amide resin and was characterized. The proteolytic stability of the D-aa KK-11 peptide was evaluated by incubating with human serum. The presence of the intact peptide after incubation with human serum for different time periods was detected using RP-HPLC and confirmed by MALDI-TOF mass analysis. Our data showed that D-aa analog of KK-11 exhibited greatly improved stability in serum for up to 5 h. The area under the curve of the peptide peak at 5 h was 97.4% of control (t=0 h), which was reduced to 45.3% by 24 h. However, peptide KK-11 with all L-amino acids reduced to an undetectable level within 30 min (data not shown). For conjugating VivoTag 680 XL dye, the peptide was modified by incorporating a β-alanine moiety generating the precursor A_{β}-KK-11. The structure and mass of A_{β}-KK-11 were confirmed by NMR (¹H and ¹³C) and MALDI-TOF [M+H]⁺ 1588.5 Da. Next, the β-alanine terminal amino group was allowed to react with the succinimidyl ester group of the reactive fluorophore (VivoTag 680 XL) under basic conditions in polar aprotic solvent, generating the fluorescently labeled peptide with extrusion of N-hydroxysuccinimide (NHS) (Scheme 1). The mass of the fluorescence-labeled VivoTag-A_{β}-KK-11 was confirmed by MALDI-TOF, showing a peak at 2823.4 Da, corresponding to [M+H]⁺.

Uptake of Select Peptides by Melanoma Cells

As shown in the fluorescence microscopy imaging (FIG. 3A), FITC-KK-11 was present in the cytoplasm of A375 melanoma cells after 30 min incubation at 0.5 μM concentration. The uptakes of FITC-labeled KK-11 and three other peptides (KK-1, KK-12, and KK-13) were further confirmed using flow cytometry analysis (FIG. 3B). Among the four tested peptides, the uptake of KK-11 by melanoma cells was the highest, and in a dose-dependent manner. At 1 μM concentration, the average fluorescence intensity was increased to more than 20-fold of FITC alone (control). KK-1 peptide also showed significantly increased uptake in melanoma cells, but to a lesser extent compared to KK-11 at the same concentration.

KK-11 Co-Treatment Significantly Enhanced the Cytotoxicity of DOX in Human Melanoma Cells

We further determined whether KK-11 enhances the drug delivery of cytotoxic DOX and nNOS inhibitor MAC-3-190 in vitro. Human melanoma A375 cells were then incubated with DOX in the presence or absence of KK-11. As shown in FIG. 3C, DOX (0.5 μM) alone reduced cell viability to 62% of control. Co-treatment with KK-11 (1 μM) significantly enhanced the cytotoxicity of DOX and decreased cell viability to 40% of control (p<0.001). At 1 μM, DOX in combination with KK-11 reduced cell viability to 14% of control cells, while 24% of melanoma cells survived from DOX treatment alone. Of note, treatment of melanoma cells with KK-11 and DOX sequentially failed to produce any changes on DOX cytotoxicity in melanoma cells.

The effects of KK-11 co-treatment (1 μM) with nNOS inhibitor MAC-3-190 was studied at different concentrations (25% IC₅₀, 50% IC₅₀, IC₅₀, and 2×IC₅₀). However, we did not observe any significant enhancement of cytotoxicity in the presence of KK-11 compared to MAC-3-190 treatment alone (FIG. 3D).

Bio-Distribution of KK-11 in Tumor Xenografts

Armed with this specific melanoma-binding peptide, we further studied the effects of KK-11 on drug delivery and antitumor efficacy. First, D-aa KK-11 was conjugated to VivoTag 680 XL fluorescence tag (Scheme 1) to visualize the in vivo distribution in athymic mice bearing human melanoma xenografts. After injection via tail vein, the mice were then taken for in vivo imaging using IVIS CT-Fluorescence imaging system. As shown in FIG. 4A, by 30 min after injection, VivoTag-KK-11 distributed to the xenograft tumor and accumulated in the spotted area. The fluorescence remained detectable in tumor xenografts 25 h after the administration of VivoTag-KK-11.

Further ex vivo studies demonstrated that the peptide distributed to the tumor and to several other organs nonspecifically within 5 min, including the lungs, heart, and spleen (FIG. 4B), but cleared readily from other organs by 6 h after injection. The ex vivo imaging of excised tumor xenografts confirmed the presence of the peptide in tumor tissue. The fluorescence of VivoTag-KK-11 was evident as early as 5 min after IV injection and remained detectable up to 24 h post injection in tumor xenografts. This finding was consistent with the in vivo imaging study in live mice (FIG. 4A). After 6 h, VivoTag-KK-11 was predominantly detected in kidneys up to 48 h after injection, where the peptide is metabolized. Of note, no brain penetration was observed. The preferential and prolonged accumulation of D-aa KK-11 in the tumor suggests it may enhance the targeted delivery of anticancer drugs to melanoma tumors, which will ultimately improve their antitumor activities.

Co-Administration of a Melanoma Targeted Peptide D-aa KK-11 Enhanced the Antitumor Activity of nNOS Inhibitor MAC-3-190

In vitro, we did not observe any significant difference in cytotoxicity between MAC-3-190 and co-administration of KK-11 and MAC-3-190 (FIG. 3D). In addition, our previous studies and preliminary observations demonstrated that MAC-3-190 (5 mg/kg/day) does not have any significant anti-tumor activity in vivo (Tong et al. Sci Rep 12:1701, 2022) Therefore, we evaluated if co-administration with KK-11 peptide would affect the antitumor activity of MAC-3-190 in vivo in mice carrying melanoma xenografts. Using an A375 melanoma xenograft mouse model, we compared the antitumor activity between mice injected with MAC-3-190 (5 mg/kg) and mice injected with a mixture of D-aa KK-11/MAC-3-190. Mice were also injected with saline or D-aa KK-11 to serve as controls. As shown in FIG. 5A, co-administration of D-aa KK-11 with MAC-3-190 (1:11 molar ratio) exhibited significant inhibitory effects after 21-day treatment on tumor growth compared to the MAC-3-190 (5 mg/kg) and control groups. Co-treatment reduced tumor growth by 28.6% (p<0.05; FIG. 5B) without remarkable systemic toxicity occurring as indicated by the body weight at the end of 21 days of treatment (FIG. 5C). MAC-3-190 was used in excess as peptide can deliver higher concentrations of the drug to the cancer site with a higher drug-to-peptide ratio (DPR). For similar reasons, a peptide-drug conjugate ANG1005 with a PDR of 3 is currently in clinical trials for paclitaxel delivery (Kumthekar et al. Clin Cancer Res 26:2789-2799, 2020). Also, the approved ADCs typically have a drug-to-antibody ratio (DAR) of 3-8 (Alas et al. J Med Chem 64:216-232, 2021).

Our study suggests that in mixtures with drugs, peptide KK-11 enhances the cytotoxicity of anticancer drugs by acting as a specific melanoma-targeting drug carrier both in vitro and in vivo. When used in vivo, D-aa KK-11 likely accumulates the anticancer drug MAC-3-190 in the tumor microenvironment and within the melanoma cells, as evidenced by significant tumor volume reduction in mice treated with D-aa KK-11/MAC-3-190 compared to mice treated with MAC-3-190 alone.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A tumor-targeting peptide comprising one of GRRPRPRPRP, GRRPRPRPRPW, GRRPRPRPRPWW, GRRPRPRPRPWWW, GRRPRPRPRPWVWW, GARPRPRPRP, GRAPRPRPRP, GRRPAPRPRP, GRRPRPAPRP, GRRPRPRPAP, VPWXEPAYQRFL, WXEAAYQRFL, EPAAYQRFTA, RVPWLEAPYA, FVPWLEAPYA, RGDAAYQRFL, RGEPAYQRFL, RGEPAYQGRFL, RGDPAYQGRFL, YHWYGTPQNVI, WQTNYIHPYVYG, YGPWYNHYITQV, AHWYGYTPQNVI, YAWYGYTPQNVI, YHAYGYTPQNVI, YHWAGYTPQNVI, YHWYGATPQNVI, YHWYGYAPQNVI, YHWYGYTAQNVI, YHWYGYTPANVI, YHWYGYTPQAVI, YHWYGYTPQNAI, YHWYGYTPQNVA, YHWYGYTPENVI, YHWYGYTPQDVI, YHWYGYTPQKVI, CLSDGKRKC, AGRKLDSKA, ADRSKGKLA, ALSDGKRKA, ALSGKRKC, CASDGKRKC, CLADGKRKC, CLSAGKRKC, CLSDAKRKC, CLSDGARKC, CLSDGKAKC, CLSDGKRAC, CLSDGKRKA, CLSEGKRKC, CLSDGRRKC, CLSDGKRRC, and WLSDGKRKC.

2. The tumor-targeting peptide of claim 1, wherein the peptide comprises GRRPRPRPRP or VPWXEPAYQRFL.

3. The tumor-targeting peptide of claim 1, wherein one or more of the amino acids are a D-amino acid.

4. A composition comprising a chemotherapeutic agent and at least one tumor-targeting peptide comprising one of GRRPRPRPRP, GRRPRPRPRPW, GRRPRPRPRPWW, GRRPRPRPRPWWW, GRRPRPRPRPWVWW, GARPRPRPRP, GRAPRPRPRP, GRRPAPRPRP, GRRPRPAPRP, GRRPRPRPAP, VPWXEPAYQRFL, WXEAAYQRFL, EPAAYQRFTA, RVPWLEAPYA, FVPWLEAPYA, RGDAAYQRFL, RGEPAYQRFL, RGEPAYQGRFL, RGDPAYQGRFL, YHWYGTPQNVI, WQTNYIHPYVYG, YGPWYNHYITQV, AHWYGYTPQNVI, YAWYGYTPQNVI, YHAYGYTPQNVI, YHWAGYTPQNVI, YHWYGATPQNVI, YHWYGYAPQNVI, YHWYGYTAQNVI, YHWYGYTPANVI, YHWYGYTPQAVI, YHWYGYTPQNAI, YHWYGYTPQNVA, YHWYGYTPENVI, YHWYGYTPQDVI, YHWYGYTPQKVI, CLSDGKRKC, AGRKLDSKA, ADRSKGKLA, ALSDGKRKA, ALSGKRKC, CASDGKRKC, CLADGKRKC, CLSAGKRKC, CLSDAKRKC, CLSDGARKC, CLSDGKAKC, CLSDGKRAC, CLSDGKRKA, CLSEGKRKC, CLSDGRRKC, CLSDGKRRC, and WLSDGKRKC.

5. The composition of claim 5, wherein said chemotherapeutic agent comprises an alkylating agent (i.e, cyclophosphamide, mechlorethamine, chlorambucil, melphalan, dacarbazine, nitrosoureas, temozolomide), an anthracycline (i.e., daunorubicin, doxorubicin, epirubicin, idarubicin, mitoxantrone, valrubicin), a taxane (i.e., paclitaxel, docetaxel, abraxane, taxotere), epithilone, a histone deacetylase inhibitor (i.e., vorinostat, romidepsin), a topoisomerase inhibitor (i.e., irinotecan, topotecan, etoposide, teniposide, tafluposide), a kinase inhibitor (i.e., bortexomib, erlotinib, gefitinib, imatinib, vemurafenib, vismodegib), a nucleoside analog (i.e., azacitidine, azathioprine, capecitabine, cytarabine, doxifluridine, fluorouracil, gemcitabine, hydroxyurea, mercaptopurine, methotrexate, tioguanine), a peptide antibiotic (i.e., bleomycin, actinomycin), a platinum-based agent (i.e., carboplatin, cisplatin, oxaliplatin), a retinoid (i.e., tretinoin, alitretinoin, bexarotene), a vinca alkaloid (i.e., vinblastine, vincristine, vindesine, vinorelbine), or a neuronal nitric oxide synthase inhibitor (i.e., MAC-3-190, HH044)

6. The composition of claim 5, wherein the tumor-targeting peptide is VPWXEPAYQRFL and the chemotherapeutic agent is doxorubicin or MAC-3-190.

7. A method of treating cancer comprising administering to a subject in need thereof at least one chemotherapeutic agent and at least one tumor-targeting peptide comprising one of GRRPRPRPRP, GRRPRPRPRPW, GRRPRPRPRPWW, GRRPRPRPRPWWW, GRRPRPRPRPWWWW, GARPRPRPRP, GRAPRPRPRP, GRRPAPRPRP, GRRPRPAPRP, GRRPRPRPAP, VPWXEPAYQRFL, WXEAAYQRFL, EPAAYQRFTA, RVPWLEAPYA, FVPWLEAPYA, RGDAAYQRFL, RGEPAYQRFL, RGEPAYQGRFL, RGDPAYQGRFL, YHWYGTPQNVI, WQTNYIHPYVYG, YGPWYNHYITQV, AHWYGYTPQNVI, YAWYGYTPQNVI, YHAYGYTPQNVI, YHWAGYTPQNVI, YHWYGATPQNVI, YHWYGYAPQNVI, YHWYGYTAQNVI, YHWYGYTPANVI, YHWYGYTPQAVI, YHWYGYTPQNAI, YHWYGYTPQNVA, YHWYGYTPENVI, YHWYGYTPQDVI, YHWYGYTPQKVI, CLSDGKRKC, AGRKLDSKA, ADRSKGKLA, ALSDGKRKA, ALSGKRKC, CASDGKRKC, CLADGKRKC, CLSAGKRKC, CLSDAKRKC, CLSDGARKC, CLSDGKAKC, CLSDGKRAC, CLSDGKRKA, CLSEGKRKC, CLSDGRRKC, CLSDGKRRC, and WLSDGKRKC.

8. The method of claim 7, wherein the cancer comprises skin cancer.

9. The method of claim 8, wherein the cancer is melanoma.

10. The method of claim 7, wherein the at least one chemotherapeutic agent is selected from an alkylating agent (i.e, cyclophosphamide, mechlorethamine, chlorambucil, melphalan, dacarbazine, nitrosoureas, temozolomide), an anthracycline (i.e., daunorubicin, doxorubicin, epirubicin, idarubicin, mitoxantrone, valrubicin), a taxane (i.e., paclitaxel, docetaxel, abraxane, taxotere), epithilone, a histone deacetylase inhibitor (i.e., vorinostat, romidepsin), a topoisomerase inhibitor (i.e., irinotecan, topotecan, etoposide, teniposide, tafluposide), a kinase inhibitor (i.e., bortexomib, erlotinib, gefitinib, imatinib, vemurafenib, vismodegib), a nucleoside analog (i.e., azacitidine, azathioprine, capecitabine, cytarabine, doxifluridine, fluorouracil, gemcitabine, hydroxyurea, mercaptopurine, methotrexate, tioguanine), a peptide antibiotic (i.e., bleomycin, actinomycin), a platinum-based agent (i.e., carboplatin, cisplatin, oxaliplatin), a retinoid (i.e., tretinoin, alitretinoin, bexarotene), a vinca alkaloid (i.e., vinblastine, vincristine, vindesine, vinorelbine), or a neuronal nitric oxide synthase inhibitor (i.e., MAC-3-190, HH044)

11. The method of claim 10, wherein the tumor-targeting peptide is VPWXEPAYQRFL and the chemotherapeutic agent is doxorubicin or MAC-3-190.


SEQUENCE LISTING (56 sequences)

SEQ ID NO: 1 - protein, 10 aa, synthetic construct
GRRPRPRPRP

SEQ ID NO: 2 - protein, 11 aa, synthetic construct
GRRPRPRPRPW

SEQ ID NO: 3 - protein, 12 aa, synthetic construct
GRRPRPRPRPWW

SEQ ID NO: 4 - protein, 13 aa, synthetic construct
GRRPRPRPRPWWW

SEQ ID NO: 5 - protein, 14 aa, synthetic construct
GRRPRPRPRPWWWW

SEQ ID NO: 6 - protein, 10 aa, synthetic construct
GARPRPRPRP

SEQ ID NO: 7 - protein, 10 aa, synthetic construct
GRAPRPRPRP

SEQ ID NO: 8 - protein, 10 aa, synthetic construct
GRRPAPRPRP

SEQ ID NO: 9 - protein, 10 aa, synthetic construct
GRRPRPAPRP

SEQ ID NO: 10 - protein, 10 aa, synthetic construct
GRRPRPRPAP

SEQ ID NO: 11 - protein, 12 aa, synthetic construct
  NON_STD 4: note = X=norleucine
VPWXEPAYQRFL

SEQ ID NO: 12 - protein, 10 aa, synthetic construct
  NON_STD 2: note = X=norleucine
WXEAAYQRFL

SEQ ID NO: 13 - protein, 10 aa, synthetic construct
EPAAYQRFTA

SEQ ID NO: 14 - protein, 10 aa, synthetic construct
RVPWLEAPYA

SEQ ID NO: 15 - protein, 10 aa, synthetic construct
FVPWLEAPYA

SEQ ID NO: 16 - protein, 10 aa, synthetic construct
RGDAAYQRFL

SEQ ID NO: 17 - protein, 10 aa, synthetic construct
RGEPAYQRFL

SEQ ID NO: 18 - protein, 11 aa, synthetic construct
RGEPAYQGRFL

SEQ ID NO: 19 - protein, 11 aa, synthetic construct
RGDPAYQGRFL

SEQ ID NO: 20 - protein, 11 aa, synthetic construct
YHWYGTPQNVI

SEQ ID NO: 21 - protein, 12 aa, synthetic construct
WQTNYIHPYVYG

SEQ ID NO: 22 - protein, 12 aa, synthetic construct
YGPWYNHYITQV

SEQ ID NO: 23 - protein, 12 aa, synthetic construct
AHWYGYTPQNVI

SEQ ID NO: 24 - protein, 12 aa, synthetic construct
YAWYGYTPQNVI

SEQ ID NO: 25 - protein, 12 aa, synthetic construct
YHAYGYTPQNVI

SEQ ID NO: 26 - protein, 12 aa, synthetic construct
YHWAGYTPQNVI

SEQ ID NO: 27 - protein, 12 aa, synthetic construct
YHWYGATPQNVI

SEQ ID NO: 28 - protein, 12 aa, synthetic construct
YHWYGYAPQNVI

SEQ ID NO: 29 - protein, 12 aa, synthetic construct
YHWYGYTAQNVI

SEQ ID NO: 30 - protein, 12 aa, synthetic construct
YHWYGYTPANVI

SEQ ID NO: 31 - protein, 12 aa, synthetic construct
YHWYGYTPQAVI

SEQ ID NO: 32 - protein, 12 aa, synthetic construct
YHWYGYTPQNAI

SEQ ID NO: 33 - protein, 12 aa, synthetic construct
YHWYGYTPQNVA

SEQ ID NO: 34 - protein, 12 aa, synthetic construct
YHWYGYTPENVI

SEQ ID NO: 35 - protein, 12 aa, synthetic construct
YHWYGYTPQDVI

SEQ ID NO: 36 - protein, 12 aa, synthetic construct
YHWYGYTPQKVI

SEQ ID NO: 37 - protein, 9 aa, synthetic construct
CLSDGKRKC

SEQ ID NO: 38 - protein, 9 aa, synthetic construct
AGRKLDSKA

SEQ ID NO: 39 - protein, 9 aa, synthetic construct
ADRSKGKLA

SEQ ID NO: 40 - protein, 9 aa, synthetic construct
ALSDGKRKA

SEQ ID NO: 41 - protein, 8 aa, synthetic construct
ALSGKRKC

SEQ ID NO: 42 - protein, 9 aa, synthetic construct
CASDGKRKC

SEQ ID NO: 43 - protein, 9 aa, synthetic construct
CLADGKRKC

SEQ ID NO: 44 - protein, 9 aa, synthetic construct
CLSAGKRKC

SEQ ID NO: 45 - protein, 9 aa, synthetic construct
CLSDAKRKC

SEQ ID NO: 46 - protein, 9 aa, synthetic construct
CLSDGARKC

SEQ ID NO: 47 - protein, 9 aa, synthetic construct
CLSDGKAKC

SEQ ID NO: 48 - protein, 9 aa, synthetic construct
CLSDGKRAC

SEQ ID NO: 49 - protein, 9 aa, synthetic construct
CLSDGKRKA

SEQ ID NO: 50 - protein, 9 aa, synthetic construct
CLSEGKRKC

SEQ ID NO: 51 - protein, 9 aa, synthetic construct
CLSDGRRKC

SEQ ID NO: 52 - protein, 9 aa, synthetic construct
CLSDGKRRC

SEQ ID NO: 53 - protein, 9 aa, synthetic construct
WLSDGKRKC

SEQ ID NO: 54 - protein, 12 aa, synthetic construct
  NON_STD 4: note = X=D-norleucine
  MOD_RES 10: note = D-arginine
VPWXEPAYQRFL

SEQ ID NO: 55 - protein, 13 aa, synthetic construct
  MOD_RES 1: note = X=FITC-beta alanine
  NON_STD 4: note = X=norleucine
XVPWXEPAYQRFL

SEQ ID NO: 56 - protein, 13 aa, synthetic construct
  MOD_RES 1: note = X=VivoTag-beta alanine
  NON_STD 4: note = X=D-norleucine
  MOD_RES 10: note = D-arginine
XVPWXEPAYQRFL